(19) United States
(12) Patent Application Publication
(10) Pub. No.: US 2012/0232430 A1
(43) Pub. Date: Sep. 13, 2012

(54) UNIVERSAL ACTIGRAPHIC DEVICE AND METHOD OF USE THEREFOR

(76) Inventors: Patrick Boissy, Sherbrooke (CA); Mathieu Hamel, Magog (CA); Simon Brière, Sherbrooke (CA)

(21) Appl. No.: 13/044,995

(22) Filed: Mar. 10, 2011

(51) Int. Cl. A61B 5/103

(52) U.S. Cl. 600/595

(57) ABSTRACT

The present disclosure relates to an actigraphic device based on a universal actigraphic platform. Parameters from a plurality of sensors are acquired and correlated for determining a profile of use of the device. The device may be installed on a vehicle, for example a powered wheelchair, or be worn by a user. Two or more devices may form a network of communicating devices worn by a user. A method of using the actigraphic device for acquiring a profile of use is also provided.

### TECHNICAL FIELD

The present disclosure relates to the field of actigraphic measurements, and more specifically, to a device and a method for acquiring actigraphic measurements.

### BACKGROUND

Mobility is broadly defined as the ability to move oneself, whether by walking, by using assistive devices such as wheelchairs, or by using transportation, within community environments that expand from one's home, to the neighborhood, and to regions beyond. It is also a fundamental part of both basic activities of daily living (ADL) and instrumental activities of daily living (IADL). Aging and diseases are associated with declines and deficits in a number of physiological systems that are essential for mobility and the performance of ADL and IADL: balance, strength, sensory detection and integration, motor coordination and cognitive processing. The accumulation of these declines can result in mobility impairments that may cause falls, dependence in performing ADL and IADL and limit social participation.

Social participation is an important modifiable health determinant and a key outcome measure as well as a common emerging intervention goal of health professionals. The concept of social participation can be defined as a person's involvement in a life situation or so-called social activity. A social activity is by definition carried out with others as interactions between people, with physical, social, and attitudinal environments. Interpersonal communication is a key factor in creating social interaction. These declines can be accelerated by musculo-squeletal and neurological diseases such as osteoarthritis, stroke, spinal cord injuries, Alzheimer's and Parkinson's disease (PD).

New models of disability recognize that disablement is a dynamic process, subject to change, and influenced by both intrinsic factors within the individual and extrinsic factors such as physical and social features within the environment and assistive technologies. For example, in community-dwelling older adults and residents in long-term care environments, mobility assistive devices, such as canes, walkers, manual and powered wheelchairs, facilitate independent mobility and improve the individual's ability to engage in meaningful life activities. Preserving mobility has become a more critical part of maintaining function and preventing further disability in these individuals. Understanding determinants of mobility disability and their evolution in time is essential to developing interventions aimed at preserving mobility in older adults.

The determinants of mobility disability have traditionally been studied using outcomes from laboratory (motion analysis), clinical (observational) and community (self-report) approaches. Laboratory research typically encompasses analyzing 3D whole-body motion by optically tracking during a variety of mobility tasks and activities of daily living, markers associated with the body segments of interest. Clinical measures use time metrics and qualitative scales to measure the performance of an individual on different mobility tasks. Self-report questionnaires are based on subjective perception and evaluation of the participant's capacities to perform mobility tasks and its frequency.

Although laboratory approaches to the study of mobility helps us understand some determinants of mobility disability in a controlled environment the impact of intrinsic and extrinsic obstacles described in laboratory research is not the same as that observed when a person is navigating within their home or community or using assistive technologies to maintain their mobility. Clinical measures of mobility functions, such as fixed-distance or fixed-time walking tests, mimic mobility efforts of everyday life but do not always reflect the true nature of mobility restriction. This is due to their nature of being indirect assessments (i.e. an evaluation of functional capability under controlled experimental conditions) of a real-world enacted function that is modulated by complex interactions between internal physiologic capacity, motivation, and external challenges older adults experience in daily life. Self-reports often contain subjective noise produced by recall problems or the respondents' tendencies to give “socially desired” answers. They often have satisfactory screening properties but offer little sensitivity to change or link to actual measured performance. Overall, these approaches have trade-offs in terms of precision/accuracy, validity/reliability, time/cost, training/expertise, participant burden and real-world generalization. They remain a proxy of the mobility and activities of the individual and often fail to capture the dynamics between the environment, the intrapersonal factors of mobility and activity restriction and the real life expression of this mobility and activities.

With motion sensor technologies such as accelerometers, gyroscopes, and magnetometers becoming more robust, smaller, and cheaper, there is an opportunity to directly measure the motion of an individual or object.

Advances in geotracking, including Global Positioning System (GPS) and geocoding via Global Information System (GIS) methods have allowed interested parties to efficiently track and model behaviors such as out-of-home mobility in the time-space domain and measure access to built-environment resources and exposure to social problems or risks. In recent years, the measure of an individual's lifespace has been proposed as a better way to capture both the functional and psychological aspects of mobility while offering a better reflection of actual mobility performance. Lifespace can be defined as the size of the spatial area a person purposely moves through in daily life, as well as the frequency of travel within a specific time frame. The measure thus not only captures the actual spatial extent of movement but also the desire for movement and being involved in the larger social environment. As such, a constricted life space may be a consequence of poor health. Impaired sensory, motor, or cognitive functioning make it difficult to move around in the community at the same level as before onset. Commonly diagnosed illnesses such as heart disease, neurological disease, arthritis/neuralgia, dementia, or eye disease, have been found to be associated with a smaller lifespace.

A full assessment of a one's mobility capabilities requires a very large amount of data related to the person, their behavior and their environment. Current technologies using optical and magnetic tracking allow capturing bodily movements, for example for making movies or video games and for studying biomechanics of motion. Systems based on these technologies generally use expensive systems and are for all practical purposes limited to laboratory uses. Still, these systems do not possess the capacity to record data such as bodily movements during ambulation under free living conditions over sufficiently long periods of time to provide a sufficient assessment of a person's mobility capabilities in their natural environment. Furthermore they are mostly limited to capturing motion data.

Other technologies are used in assisting personal physical training. A processor coupled with an accelerometer may for example estimate, using step counts, an energy expenditure of a user as they go through their daily activities.

Collecting data in natural environments under free living conditions over long period of times—covering several days for example—is possible, but there exist no system having sufficient data recording capability from multiple sources of sensors, coupled with a proper set of metrics, fusing this data, for establishing an actigraphic profile of a user.

### SUMMARY

Therefore, according to the present disclosure, there is provided an actigraphic device, comprising a motion sensor, an environment sensor, a processor and a memory. The motion sensor is for detecting a parameter related to a movement of the device. The environment sensor is for detecting a parameter related to an environment of the device. The processor is for determining a profile of use of the device by correlating the parameter related to the movement of the device and the parameter related to the environment of the device. The memory is for recording the profile of use.

According to the present disclosure, there is also provided a method of acquiring a profile of use of an actigraphic device. A parameter related to a movement of the device is detected. A parameter related to an environment of the device is also detected. The profile of use of the device is determined by correlating the parameter related to the movement of the device and the parameter related to the environment of the device. The profile of use is stored in a memory.

The foregoing and other features will become more apparent upon reading of the following non-restrictive description of illustrative embodiments thereof, given by way of example only with reference to the accompanying drawings.

### BRIEF DESCRIPTION OF THE DRAWINGS

Embodiments of the disclosure will be described by way of example only with reference to the accompanying drawings, in which:

FIG. 1 is a block diagram showing an exemplary architecture of a universal actigraphic platform and its components;

FIG. 2a is an illustration of a universal actigraphic device, adapted as an example of user wearable device;

FIG. 2b is a detailed view of the device of FIG. 2a;

FIG. 3 is a top view and a bottom view of internal components of the universal actigraphic device of FIG. 2a;

FIG. 4 is a block diagram of exemplary components of the universal actigraphic device of FIG. 2a;

FIGS. 5a and 5b show an example sequence of steps executed in universal actigraphic device;

FIG. 6 is a block diagram of a fusion process for use in an actigraphic device;

FIG. 7 is a perspective view of a powered wheelchair, also showing examples of various sensors and data logging components;

FIG. 8 is a perspective view of the powered wheelchair of FIG. 7, also showing axes for inertial measurements and sample data therefor;

FIG. 9a is a first geographical map showing lifespace data recorded over 5 days;

FIG. 9b is graph showing activity data recorded over 5 days; and

FIG. 10 is a second geographical map showing lifespace data recorded over 7 days.

### DETAILED DESCRIPTION

Earlier methods and devices used for obtaining actigraphic measurements did not provide long term recording of a plurality of static and dynamic parameters related to a user mobility and activity and information about their surrounding environment. They either did not integrate a variety of sensors or the possibility to add information obtained from external sensors. Some platforms did not have a compact size and were not readily portable. Earlier platforms could only be operated with extensive training.

Drawbacks of earlier actigraphic methods and devices are overcome in an actigraphic device described in the present disclosure. The actigraphic device: comprises a motion sensor, an environment sensor, a processor and a memory. The motion sensor detects a parameter related to a movement of the device. The environment sensor detects a parameter related to an environment of device. The processor determines a profile of use of the device by correlating the parameter related to the movement of the device and the parameter related to the environment of the device. The memory records the profile of use.

The platform comprises internal sensors and may receive and, use parameter data from external sensors. The platform is small and light, and is easy to use. For example, plugging the platform to a charger is simple while transferring data between the platform and external nodes is seamless. Features of the platform and a fusion of measurements from its sensors allow the determination of the activity or behavior of the user and the establishment of an actigraphic profile of a user, this profile being adaptable to represent the activity or behavior of the user over time.

A device built on this platform may also be adapted for installation on vehicles, for example on wheelchairs and automobiles, or on mobile robots. The platform may also be used as a wearable device that may, for example, be worn on an arm, a hip, a leg, or on the trunk of a user. When used as a wearable device, a plurality of platforms may form a network capable of processing static and dynamic parameters related to various segments of a user's body or external devices manipulated by the user. As a non-limiting example, two devices worn above and below the knee of a user may provide information for characterizing joint range of motion during the performance of various activities.

Disclosed herein are a universal actigraphic platform, a device built on this platform and a method using this device. The universal actigraphic platform has an open architecture for measuring, processing, recording and/or transmitting ambulatory data generated by internal or external sensors. Sensors, for example accelerometers, gyroscopes, magnetometers, and/or GPS receivers, are integrated on a small-scale printed circuit board (PCB), having for example a 1.5×2 inches size. This integration allows the creation of a wearable device that a user may wear, for example as a bracelet, in order to establish their actigraphic profile over an extended period of time. The universal actigraphic platform may be configured using parameters related to an activity or performance that is being monitored. Examples may include capture motion and environmental information through a variety of sensors. The platform, and devices built on this platform, may accept various attachment means suited for various applications.

In an embodiment, the platform is a highly miniaturized electronic data acquisition device that may be used to monitor, from a variety of locations on a subject's body or on objects within the subject's environment, signals from a plurality of sensors, including biometric sensors.

Biometrics sensors may gather biometric data relating to various physical characteristics, positions, performances, behaviors and properties of the subject or object being monitored. Biometric data includes biomechanical, biomedical and behavioral data, and may include a broad range of data types. Data may relate to a trajectory, speed, acceleration, position, orientation, and the like, of a subject's appendage, body part or object being manipulated by the subject. Data may also relate to a heart rate, oxygen saturation, respiration rate, blood pressure, temperature and/or galvanic skin response of a subject.

Other sensors may provide data may further show a posture or other status of a subject or object, indicating for example whether the subject is in a prone or erect position, whether they are moving or not. Additionally, data may show proximity of the subject to objects or people and whether or not a subject is talking to people or whether people are talking to them. The above examples are non-limiting.

The sensors may include one or more of the following technologies: accelerometer technology for detecting accelerations and decelerations, gyroscope technology that detects changes in orientation and angular velocities, compass or magnetic technology that senses position and/or alignment with relation to magnetic fields, satellite-based GPS technology, audio sensing technology with voice activity detection (VAD), radio-frequency technology, and the like. Those of ordinary skill in the art will readily determine that other types of sensor technologies may be integrated with the universal actigraphic platform disclosed herein.

An embodiment of an electronic data acquisition device is disclosed. The electronic data acquisition device comprises at least one sensor configured to associate with an activity or behavior being monitored and output data relating to the activity or behavior of the user. The present disclosure includes a microprocessor that captures data from various sensor inputs and analyzes the data for real-time feedback or for post-processing analysis using a communication module that may either store the data to memory or transmit it to a computer using radio frequency or other means of communication.

According to the present disclosure, there is also provided a method of recognizing and classifying activity and behavior derived from acquired biometric signals. Using signal processing techniques combined with heuristics and stochastic methods, biometric signals may be processed to provide recognition of the activity or behavior of a subject and establish a profile of occurrence in time of such activity or behavior.

Broadly, a universal actigraphic device may be used for a large number of applications related to telemedicine, rehabilitation, biomechanics, arts, robotic devices, healthcare research and industrial research. This list of application fields is exemplary and is not intended to limit the present disclosure.

It is possible to use this device to record measurements over a period of several weeks. Using precision sensors, the device is capable of estimating with accuracy its position within a tri-dimensional (3D) space while also recording its geographical position, in terms of altitude, longitude and latitude. Its open and flexible architecture allows its connection to external sensors, for example an electrocardiograph (ECG), an electromyograph (EMG), a respiratory belt, a stress gauge, a matrix of force sensing resistor (FSR), a voice activity detection (VAD) sensor, and the like. In an embodiment, the processor may sample some or all of the sensors at a different sampling rate to accommodate the activity or behavior being monitored while also accommodating the implementation characteristics of the various sensors. The device may further integrate additional features, for example wired or wireless communication with other similar devices or with a computer, an integrated battery charger. In an embodiment, wired communication takes place over a universal serial bus (USB) interface. In another embodiment, wireless communication may take place over an IEEE 802.15.4 communication protocol, known to those skilled in the art as Zigbee®, or over an IEEE 802.15.1, known to those skilled in the art as Bluetooth®.

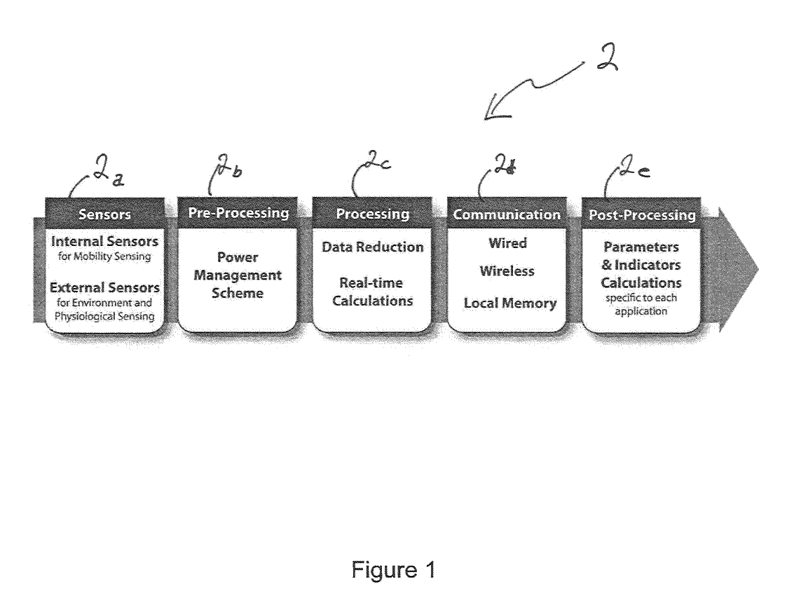

Referring now to the Drawings, FIG. 1 is a block diagram showing an exemplary architecture of a universal actigraphic platform and its components. An actigraphic platform 2 uses information from sensors 2ₐ, which may be internal or external to the actigraphic platform 2. The platform 2 has a pre-processing power management scheme 2_{b}, for ongoing verification of its internal battery (not shown but shown on later Figures). Processing 2_{c} of information received from the sensors 2ₐ includes real-time calculations, leading to a reduction of an amount of gross data to a lesser number of processed, information elements. Processed information elements may comprise a profile of use of the platform 2. The platform 2 is capable of communicating 2_{d} the processed information elements and/or the gross sensor information, either through wired or wireless connections. Alternatively or in addition, the platform 2 may store the processed information elements and/or the gross sensor information in an internal memory (not shown but shown on later Figures). Post-processing 2ₑ of the processed information elements and/or the gross sensor information may involve calculations of various parameters and indicators, according to the needs of a relevant application.

An embodiment of the universal actigraphic platform 2 may comprise a wireless (W) communication capability, an inertial measurement unit (IMU) and a GPS receiver. This platform may be thus conveniently named a “WIMU-GPS” platform. The WIMU-GPS platform may record and/or transmit to an external node a variety of actigraphic measurements and processed representations of those actigraphic measurements. Exemplary references to actigraphic devices and platforms will be made hereinbelow using the name WIMU-GPS for purposes of simplicity. Those of ordinary skill in the art will appreciate that embodiments of universal actigraphic platforms and devices may or may not comprise wireless communication or an IMU or a GPS receiver. References made herein to the WIMU-GPS should be understood as exemplary. The name WIMU-GPS as used herein is not meant to limit the scope of the present disclosure.

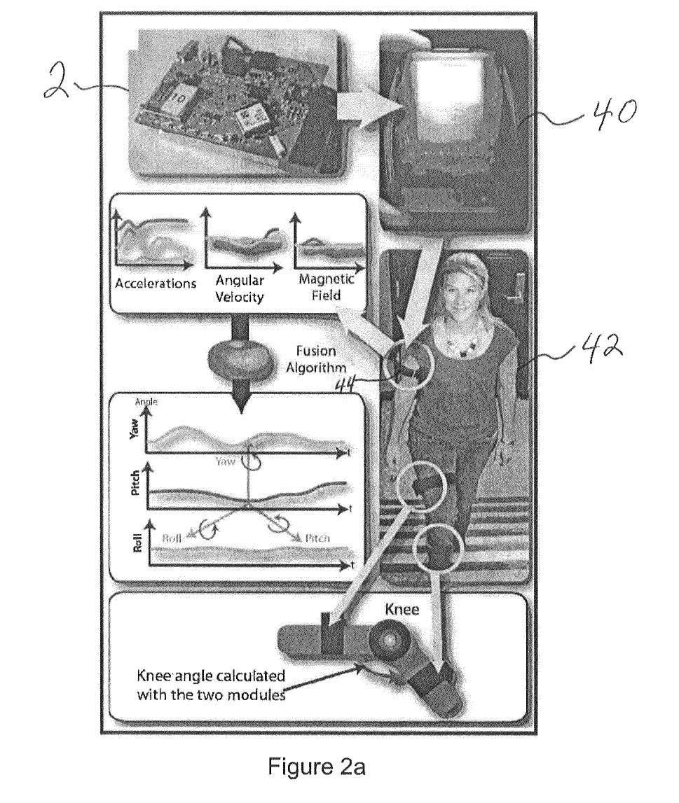

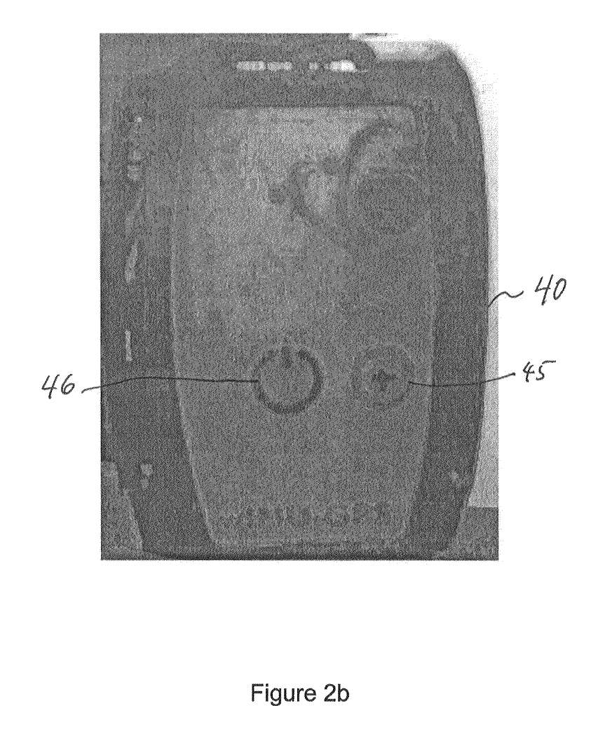

FIG. 2a is an illustration of a universal actigraphic device, adapted as an example of user wearable device. FIG. 2b is a detailed view of the device of FIG. 2a. Referring at once to FIGS. 2a and 2b, the universal actigraphic platform, or WIMU-GPS 2, which is introduced in the foregoing description of FIG. 1, is integrated in a convenient wearable device 40 adapted to be worn by a user 42. The wearable device 40 may comprise an elastic band 44 for attaching to an arm, to a leg or to other body segments of the user 42.

In the context of a wearable device 40, one or more sensors of the WIMU-GPS 2 are used as motion sensors, which may alternatively be called mobility sensors. The motion sensors may be used for detecting one or more parameters related to a movement of the device and of its user. An IMU within the wearable device 40, which comprises the WIMU-GPS 2, worn on a leg of the user 42, then acts as a motion sensor. Likewise, one or more sensors of the wearable device 40 are used as environment sensors for detecting one or more parameters related to an environment of the device and its user 42, for example a location of the user 42. For example, when the user 42 is walking outdoors, the GPS receiver may detect a change of location. In the case where the user 42 is walking outdoors, wearing the device 40 on its leg, the IMU may at the same time act as a motion sensor to provide parameters related to movements of the leg relative to the user's body and as an environment sensor to provide other parameters related to movements of the user 42 relative to her environment. The GPS receiver may also act as an environment sensor.

A user interface of the wearable device 40 may comprise push-buttons 45 and 46. The push-buttons 45 and 46 may be used to generate time stamps related to user generated events. For example, the user 42 may use the push-buttons 45 and/or 46 to record a precise time when a medication is taken.

Within the wearable device 40, a processor (shown on later Figures) determines a dynamic representation of an activity of the user 42 by using a fusion process to correlate the one or more parameters related to the movement of the device 40 worn by the user 42 and the one or more parameters related to the environment of the device 40 and of its user 42. This correlation provides a profile of use of the device 40. The processor stores the profile of use in a memory and may also store some or all of the parameters in the memory. As a non-limiting example, the processor may estimate a location position or a geographical location of the user 42 while also determining the body angles, the range of movements (ROM), the walking speed, the bodily position (sitting, standing, laying down), and combine those elements.

The wearable device 40 may further comprise a biometric sensor for detecting a physical parameter of the user 42. Its processor may thus determine the profile of use on the added basis of the physical parameter.

The exemplary wearable device 40 is built on the WIMU-GPS platform 2, which may comprise an external input/output (I/O) port, a USB connector and/or a wireless communication link (internal components being shown on later Figures). Such a communication port of the wearable device 40 may be used for outputting the profile of use as well as the parameters related to the movement of the device 40 and the parameters related to the environment of the device 40, or any other parameter. The communication port of the wearable device 40 may also be used for inputting an external sensor parameter. The processor may thus determine a profile of use of the device 40 and a dynamic representation of the activity of the user 42 on the added basis of the external sensor parameter, correlated with internal sensor parameters.

In an embodiment, the wearable device 40 stores in memory subsequent instances of the profile of use and of the various sensor parameters. In this embodiment, the device 40 comprises a post-processor for determining an actigraphic user profile based on the instances of the profile of use of the device 40 collected over time. One possible real-life example of post-processing result and its analysis may comprise a rapid change in a user's body angle detected concurrently with a rapid acceleration and deceleration, indicative of a fall. In another embodiment, the wearable device 40 may output the instances of the profile of use for external post-processing, for example in a computer.

A network of wearable devices 40 may be used for concurrently monitoring various movements or other characteristics of the user 42. As a non-limiting example, two wearable devices 40 placed, respectively, above and below the knee of the user 42 may calculate joint angles of the knee. A first wearable device 40 worn by the user 42 sends, via its communication port, a first set of parameters including a first profile of use representative of an activity of a first body segment of the user 42 to a second wearable device 40 worn by the user 42 on a second body segment. The second wearable device 40 receives the first set of parameters via its own communication port. The second wearable device 40 produces a second profile of use representative of an activity of the second body segment. A processor of the second wearable device 40 then determines a combined profile of use by correlating the first profile of use and the second profile of use. The first and second wearable devices 40 may communicate via wired or wireless links, depending on the communication capabilities implemented in the WIMU-GPS.

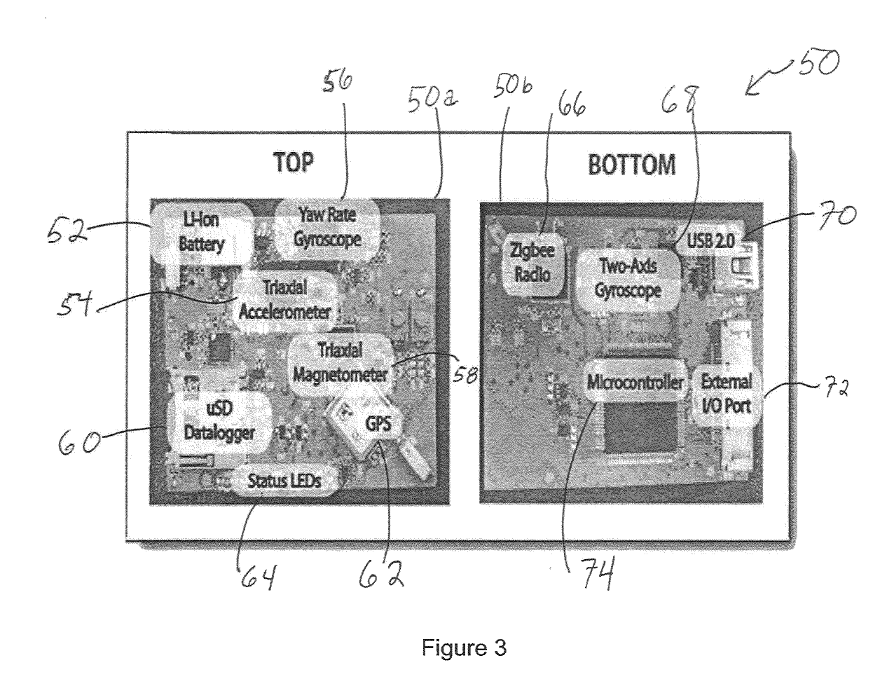

FIG. 3 is a top view and a bottom view of internal components of the user wearable device of FIG. 2a. FIG. 3 thus provides exemplary details of the WIMU-GPS platform 2 introduced in the foregoing description of FIG. 1. Of course, size and weight constraints associated with the wearable device 40 may differ from those associated with device installed on a vehicle such as, for example, on a powered wheelchair. Consequently, those of ordinary skill in the art may select distinct physical embodiments for distinct applications. FIG. 3 shows two sides 50a and 50b of a printed circuit board (PCB) 50. Alternatively, two distinct PCBs 50a and 50b may be used, connecting with each other via a flexible flat cable or via any suitable means. The exemplary PCB 50 supports a battery 52, which may be a Lithium-ion battery, a triaxial accelerometer 54 capable of detecting acceleration and deceleration over a 3D space, a yaw rate gyroscope 56, a triaxial magnetometer 58, a memory 60, for example a NSD card used as a datalogger, a GPS receiver 62, which in an embodiment is a SiRF Star III GPS receiver, one or more status light emitting diodes (LED) 64, a wireless communication link 66, which may be a Zigbee® unit, a two-axis gyroscope 68, a wired communication port 70, for example a USB version 2.0 connector, an external I/O port 72, which may be a serial port or a parallel port, and a microcontroller 74 used as a general processor or post-processor. In an embodiment, the microcontroller 74 is a MSP430F5438 unit from Texas Instruments Incorporated. The microcontroller 74 may receive parameter values from any of the sensors 54, 56, 58, 62 and 68 and from external sensors (not shown), these parameter values being received by any of the wireless communication link 66, the wired communication port 70 and/or the external I/O port 72, and presented to the microcontroller 74. The microcontroller 74 may also receive inputs related to user generated events from the push-buttons 45 and/46. The microcontroller 74 correlates the parameter values, and possibly the user generated events, and calculates a profile of use of the wearable device 40. The microcontroller 74 stores the profile of use in the memory 60 and may further store some or all of the parameter values for later post-processing. The microcontroller 74 controls sending of information, for example parameter values and the profile of use, to outside receivers (not shown) via any one of the wireless communication link 66, the wired communication port 70 and/or the external I/O port 72. Of course, the PCB 50 may further comprise other elements as is well-known to those of ordinary skill in the art.

The layout of the PCB 50 as shown on FIG. 3 is exemplary and various other layouts may be contemplated. Not all of the communication means 66, 70 and 72 may be present in some embodiments and addition or substitution of other communication means may be made. Likewise, not all of the sensors 54, 56, 58, 62 and 68 may be present and other types of sensors may be added or substituted to those shown on FIG. 3. The accelerometer 54, the gyroscopes 56 and 68, the magnetometer 58, the GPS receiver 62 and any combination thereof may be used as motion sensors of the wearable device 40. The same components or a combination thereof may be used as environment sensors of the wearable device 40.

As shown, a combination of the yaw rate gyroscope 56 with the two-axis gyroscope 68 provides a 3D gyroscope functionality. Other embodiments may comprise a 3D gyroscope implemented as a single module. Owing to the 3D capabilities of the triaxial accelerometer 54, of the triaxial magnetometer 58 and of the combined features of the yaw rate gyroscope 56 and of the two-axis gyroscope 68, a dynamic 3D representation of an activity of a user of the wearable device 40 may be determined by the microcontroller 74.

The microcontroller 74 may implement a fusion process, which may further involve the use of a Kalman filter, of a neural network, of fuzzy logic, or of any similar filtering process. The fusion process may estimate orientation values of the WIMU-GPS relative to a fixed frame of reference, based for example on gravitation and/or on the magnetic north. These orientation values may be obtained in all three (3) dimensions (yaw, pitch, roll) for the wearable device 40 positioned on a segment of the user's body. The fusion process may further correlate various parameter values of different types, from various internal and/or external sensors, to provide a profile of use of the wearable device 40 and, more broadly, a dynamic representation of an activity of the user of the wearable device 40. Using multiple wearable devices 40 and linking them via their communication means allows computing of the kinematics of a specific body segment, for example a knee. This may be done for any body segment and between body segments.

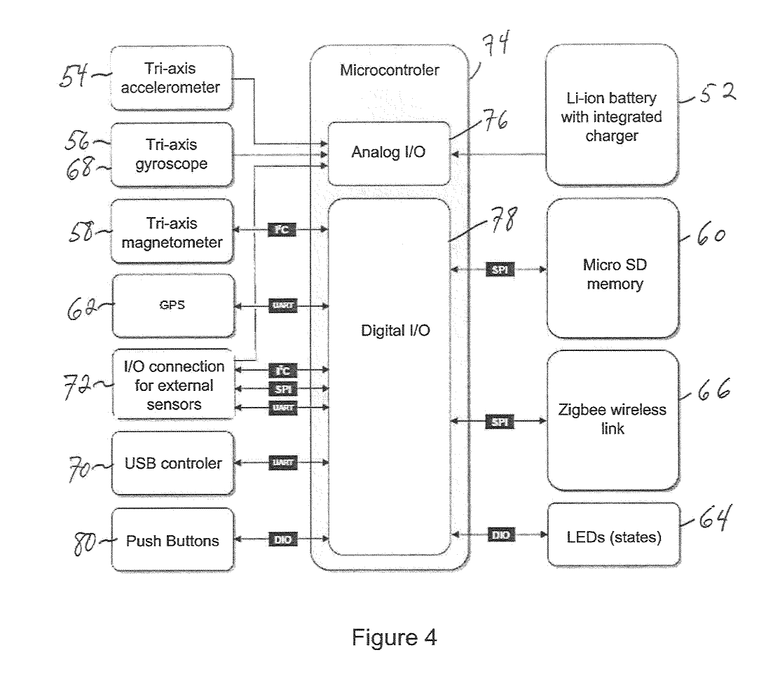

FIG. 4 is a block diagram of exemplary components of the user wearable device of FIG. 2a. FIG. 4 provides another exemplary view of the WIMU-GPS platform 2, in schematic form. It shows that the microcontroller 74 comprises an analog input/output (I/O) 76 for receiving a voltage measurement from the Lithium-ion battery 52 and its integrated charger (not specifically shown), to the tri-axis accelerometer 54, to the gyroscopes 56 and 68, and to the external I/O port 72. The microcontroller 74 also comprises a digital I/O 78 for communicating with the memory 60, with the wireless communication link 66 and with the external I/O port 72 via serial peripheral interfaces (SPI), with the one or more status LEDs 64 and with push-buttons 80 via digital input/output (DIO) interfaces, with the tri-axis magnetometer 58 and with the external I/O port 72 via inter integrated circuit (I²C) interfaces, and with the GPS receiver 62, with the external I/O port 72 and with the USB controller via a universal asynchronous receiver/transmitter (UART). The layout of FIG. 4 is exemplary as the universal actigraphic platform may comprise more or less elements than those illustrated. Those of ordinary skill in the art will be able to substitute one type of interface, for example connecting a given sensor to the microcontroller 74, with another type of interface.

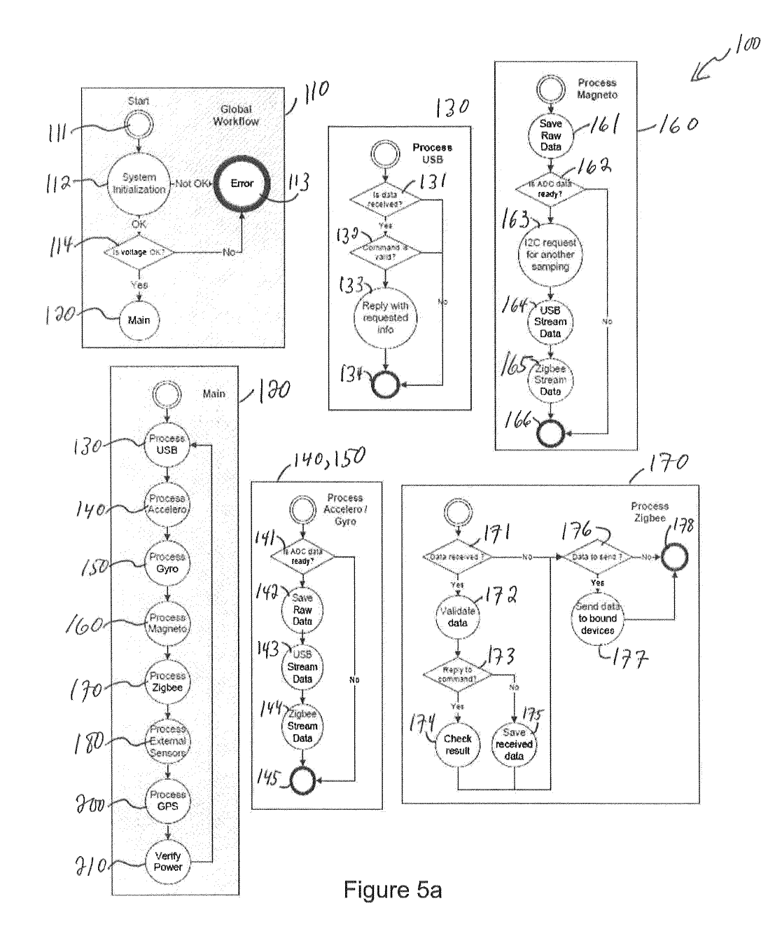

FIGS. 5a and 5b show an example sequence of steps executed in an actigraphic device. Acquisition of a profile of use of the actigraphic device may lead to the production of an actigraphic profile of a user. This may be applicable, for example, to a user wearing the actigraphic device. A flow 100 of FIGS. 5a and 5b, details actions occurring within the device. The flow 100 is initiated in an initial sequence 110, upon initial start 111, for example at power on. A system initialization process 112 takes place, after which an error 113 is displayed, for example by using one of the LEDs 64, if the initialization 112 fails. Otherwise, a voltage of the battery 52 is verified 114. If the voltage is not within its specified range, an error 113 is displayed, for example using another one of the LEDs 64. If the initial sequence 110 is successful, detection of parameters at various sensors takes place in a round-robin fashion generally shown in a main sequence 120.

The main sequence 120 comprises a suite of routines 130-210, in which components of the actigraphic device are polled. Some of the routines involve sending and/or receiving parameter values, processed data and other information through various communication means of the actigraphic device. Some other routines involve detecting one or more parameters related to a movement of a user and detecting one or more parameters related to a location of the user in relation to an environment.

The main sequence 120 comprises a routine 130 for processing the USB connection 70. A test is made to determine whether or not data is received (131). If data is received, a next step determines whether or not the command is valid (132). If so, a reply to is sent on the USB connection 70 with requested information (133). The routine 130 ends (134) and the main sequence 120 continues with a routine 140 for processing parameters having potentially been received from the accelerometer 54. If analog data requiring analog to digital conversion (ADC) is received (141), the data is saved in raw format (142) and may be placed in a buffer for forwarding externally on the USB connection 70 (143) and/or in a buffer of the wireless communication link 66 (144). The routine 140 ends (145) and the main sequence 120 continues with a routine 150 for processing the gyroscopes 56 and 68. The routine 150 is identical to the routine 140, save for the different sensor as a source of parameters. A routine 160 involves processing parameters having potentially been received from the magnetometer 58. Raw data may be received on the I²C interface and stored (161). If ADC data is ready (162) an I²C request is made for sampling again a magnetometer parameter (163). Data may be sent on the USB buffer (164) and/or on the wireless communication buffer (165). The routine 160 ends (166) and the main sequence 120 continues with processing of the wireless communication link 66 in routine 170. Data may have been received (171), in which case the data is validated (172). If received data constitutes a reply to a command (172), the reply is checked (174). Otherwise, the received data comprises, for example, a parameter received from an external sensor; this data is saved in memory (175). Whether or not data had been received, it is verified whether or not there is data to be sent externally (176). If so, the data is sent to external sensors, post-processors, other actigraphic device, and the like (177). The routine 170 ends (178) and the main sequence 120 continues with processing of external sensors in a routine 180. Parameter data from one or more external sensors may be used to complement the determination of a profile of use of the actigraphic device. The routine 180 involves polling various sensors connected to the actigraphic device via various types of communication interfaces. In an exemplary sequence, sensors connected via I²C (181), SPI (183), UART (186), digital I/O (189) and analog I/O (191) are polled. On some interfaces, a request is made for a sample and a sample is received, as shown at steps 182, 184, 185, 187 and 188. On other interfaces, data is simply received. In the case of parameters received via digital I/O, process and control of the information is made (190). In the case of parameters received via analog I/O, verification is made whether ADC data is ready (192). Acquisition of parameters from the external sensors may be made using specific drivers for communication with each distinct sensor type. When parameters have been acquired, their raw data is saved in memory (192), placed on the USB buffer (194) and on the wireless communication buffer (195). The routine 180 ends (196) and the sequence 120 continues with a routine 200 for processing the GPS receiver 62. Verification is made whether or not GPS data is received (201). If so, GPS time is compared with a time indicated by an internal clock of the actigraphic device (202). If required, the internal clock is adjusted to correspond to the GPS time (203). Raw data from the GPS receiver 62 is stored (204), placed on the USB buffer (205) and on the wireless communication buffer (206). The routine 200 ends (207) and the sequence 120 continues with a routine 210 for verifying a power level of the battery 52. If the battery level is sufficient (211) and if the battery is not being charged (212), the actigraphic device may be used. Battery power data is saved in memory (213) and the routine 210 ends (217). If the battery level is not sufficient (211) or if the battery is being charged (212), the actigraphic device enters a low-power mode (214) and remains in a sleep mode while the battery power remains at a low level or while the battery is charging (215). The low-power mode ends when the battery reaches a sufficient power level and is disconnected from its charger (216), after which the routine 210 ends (217). The main sequence 210 may then resume its cycle.

Those of ordinary skill in the art will appreciate that the sequence of events shown on the flow 100 of FIGS. 5a and 5b may be extensively modified, for example by adding or removing some events or by changing the order of the events. Such modifications are within the scope of the present disclosure.

Processing of the various parameters, including fusion and correlation of the parameters, for determining a profile of use of the actigraphic device is not explicitly shown on FIGS. 5a and 5b. This processing, which is described in other parts of the present disclosure, may take place continuously within the main sequence 120, or may alternatively take place once per cycle of the main sequence 120 or once per every few cycles of the main sequence 120, as appropriate for the needs of the application. Likewise, the profile of use may be post-processed internally or may be sent towards an external post-processing unit, via any of the communication means of the actigraphic device, either on a continuous basis or at regular intervals, as appropriate for the needs of the application.

The flow 100 of FIGS. 5a and 5b has been described in relation to the actigraphic device based on the same or similar WIMU-GPS platform 2 as introduced in the preceding Figures. The flow 100 may be implemented in an actigraphic device intended for use as a wearable actigraphic device, for installation on a powered wheelchair or on another vehicle, or for similar uses.

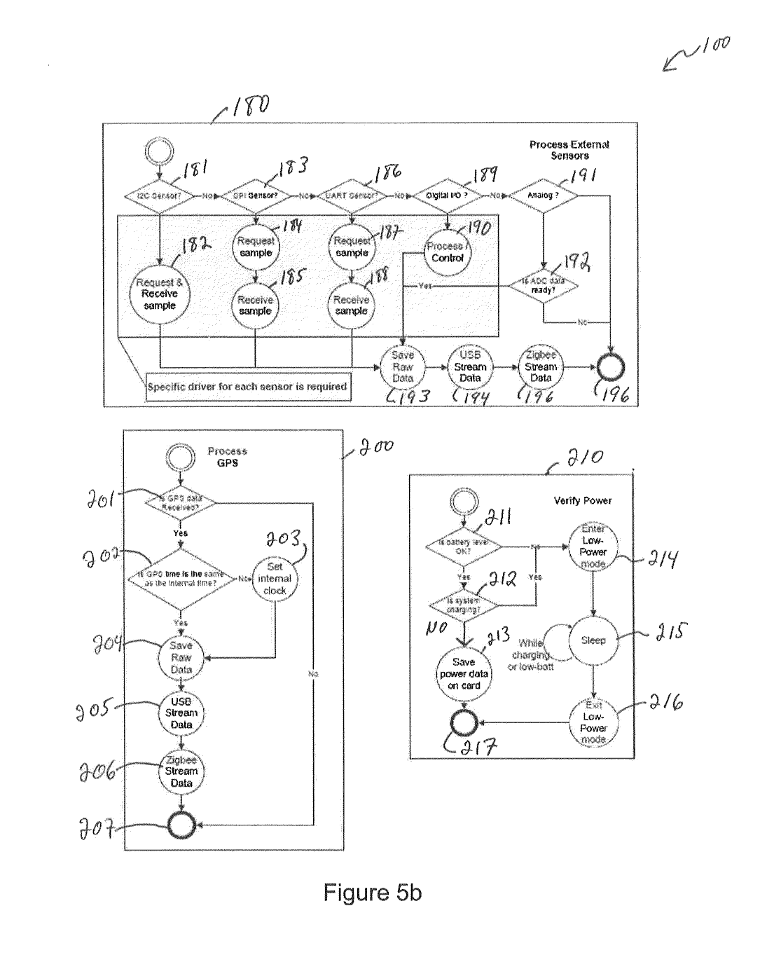

FIG. 6 is a block diagram of a fusion process for use in an actigraphic device. A fusion process 220 determines a dynamic representation of an activity of a user of the wearable device 40 or a dynamic representation of another use of the WIMU-GPS platform. The fusion process 220 may implement a Kalman filter, a fuzzy logic process, a neural network or any suitable process for correlating and filtering parameters from various sensors, with the goal of determining a profile of use of the actigraphic device. The fusion process 220 may mitigate random variations in the detected parameters. In particular, the fusion process 220 may estimate by prediction the profile of use of the actigraphic device while compensating for magnetic perturbations, transient variations in acceleration parameters, sensor imprecision and ambient noise. The fusion process 220 may be implemented in the microcontroller 74 or may be implemented as a distinct component of the WIMU-GPS 2, for example a co-processor, the distinct component (not shown) being coupled with the microcontroller 74. The fusion process 220 receives sensor parameters from the accelerometer 54, from the gyroscopes 56 and 68, and from the magnetometer 58. The fusion process 220 may also receive parameters from other sensors. Calibration parameters 222 may reside in the memory 60 or may be received at the microcontroller 74 from any one of the sensors 54, 56, 68 and/or 58. The calibration parameters 222 are provided to the fusion process 220, via any one of the various communication means of the WIMU-GPS platform 2, for adjusting a sensor modelization module 224. Parameter values received from the sensors 54, 56, 68 and 58 are first adapted by the sensor modelization module and are applied to a measurement updating module 226. The measurement updating module 226 uses sensor gains and noise filtering parameters for processing the sensor parameters and for calculating a profile of use of the actigraphic device. In the specific example of FIG. 6, wherein parameters are obtained from the accelerometer 54, the gyroscopes 56 and 68 and from the magnetometer 58, the fusion process 220 provides a dynamic representation of an orientation of the actigraphic device. The dynamic representation 228 is also applied via a feedback loop 232 to a timing update module 230. The timing update module 230 uses state estimation and covariance propagation to provide noise and bias values to adjust the sensor modelization unit 224. The timing update module 230 provides timing information to the measurement updating unit 226.

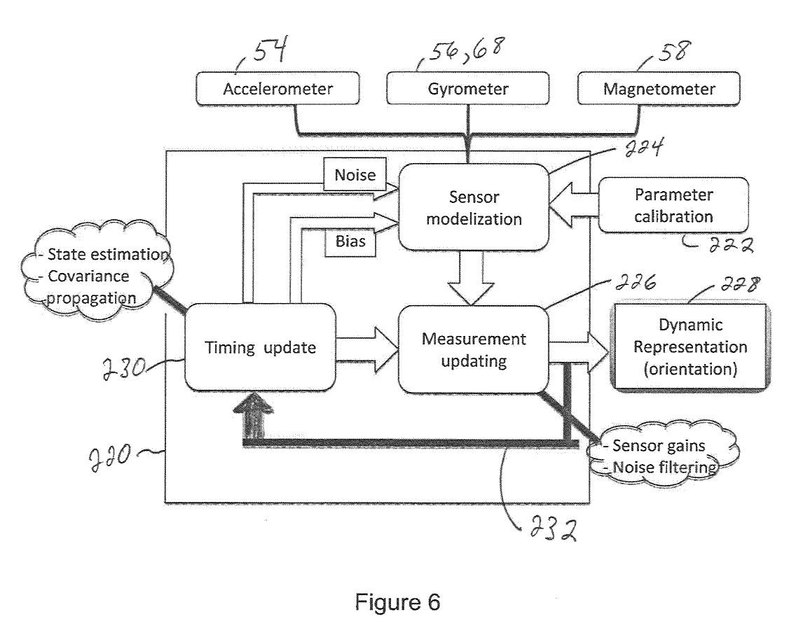

The device universal actigraphic platform introduced hereinabove may be used, in combination with external sensors, to provide a profile of use of a device placed on a powered wheelchair (PW). The WIMU-GPS 2 may thus provide a measure of a PW usage profile. A user's PW is instrumented with a WIMU-GPS 2, which includes embedded sensors, and with external sensors. Sensor data and fusion correlation processes are used to extract a profile of use, providing a dynamic representation of an activity of the PW from recorded data. The dynamic representation is used as a variable to characterize PW use. FIG. 7 is a perspective view of a powered wheelchair, also showing examples of various sensors and data logging components. An actigraphic device based on the WIMU-GPS platform 2, comprising a datalogger and embedded sensors, may be installed on a PW 4, for example by a technician. The WIMU-GPS 2 generally comprise similar features as those introduced in the description of earlier Figures and may thus provide or receive similar sensor parameters and process them to produce similar dynamic representations of an actigraphic profile of a user of the PW 4. Alternatively, the WIMU-GPS 2 may be mounted on various other types of vehicles, when there is a desire to record dynamic data related to users of such vehicles.

The WIMU-GPS 2 doesn't interfere with any of the PW 4 functions and may record data autonomously, without any user intervention, over extended periods of time, for example over 21 days. The WIMU-GPS 2 may comprise internal sensors and may also connect with external sensors. External sensors may be connected to the WIMU-GPS 2 via an I/O control box 16. For limited mobility use, for example when a user of the PW 4 is indoors, an external sensor may be stationary and wirelessly provide its parameters to the WIMU-GPS 2. The sensors may include one or more motion sensors for detecting one or more parameters related to a movement of the PW 4 on which the actigraphic device is mounted, one or more environment sensor for detecting one or more static or dynamic parameters related to an environment of the actigraphic device and of the PW 4, and may further include one or more user behavior sensors for detecting one or more static or dynamic parameters of a user of the actigraphic device and of the PW 4. In an embodiment, sensors embedded within the WIMU-GPS 2 include a 3D gyroscope, a 3D accelerometer, a 3D magnetometer, and/or a GPS receiver, which may be used as motion sensors. The 3D accelerometer and/or the GPS receiver may also be used as environment sensors. The WIMU-GPS 2 may further comprise a battery-charging sensor and other well-known components (components of the WIMU-GPS 2 are not shown on FIG. 7, but are shown on earlier Figures). These sensors record various, generally dynamic parameters of the PW 4 such as angular speed and orientation of the PW 4, vibration, geo-referenced position and speed of travel. Some parameters related to the environment of the PW 4, indicating for example whether the PW 4 is located indoor or outdoor, may have a more static characteristic while other environment sensors may be dynamic. Parameters may be used to detect the orientation of the PW 4, for example seat tilt angle, the ground surface type and the impacts on the chair as it travels, the community lifespace of the user, moments when they use other means of transport, and the frequency and duration of battery charging cycles.

External sensors may include a wheel encoder 14, acting as a motion sensor by counting a number of wheel revolutions to provide a measurement of a linear position and an estimation of speed and distance travelled by the wheelchair. Ultrasonic range finders, for example sonars 10, located at various positions on the PW 4, for example at the front, the back, the left, the right and the top of the PW 4, may act as environment sensors by returning the distance to the nearest obstacles, also providing indoor/outdoor discrimination, allowing a description of the environment around the PW 4. Thereby, close objects, open fields and indoor/outdoor locations may be identified. One or more cameras (not shown) may also be used as environment sensors by providing, for example, information about the nearest obstacles. A microphone combined with a microprocessor may be used as a voice activity detector (not shown), also called VAD sensor. The VAD sensor may provide information about the environment of the user of the PW 4, for example by detecting voices of surrounding persons. Recording audio logs using speech/non-speech detection in combination with other features of the activity or environment may provide a measure of the social participation of a person. This information may provide a measure of social participation of the user of the PW 4. A 3-by-3 FSR matrix 6 mounted on a Plexiglas® sheet 8 fixed under the seat may be used as a user behavior sensor by capturing shifting of a center of pressure of the user when seated, and by detecting the presence or absence of the user on the PW 4. Control signals from a joystick 12 or other control means used by the user of the PW 4 may also be captured. When the WIMU-GPS 2 is mounted on other types of vehicles, control signals may be obtained from a pedal, a steering wheel, or from any other actuator. Such control signals provide indications related to a frequency of commands, a timing of the commands, a smoothness of the commands, such behavioral parameters of the user and allowing correlation of the user's input with any outcome, such as an eventual impact against an object, for a specific environment, which may be defined as a close or open environment, with or without obstacles.

Inputs from these internal and external sensors as seen on FIG. 7 are acquired by a processor, within the WIMU-GPS 2, for use in monitoring a profile of use of the actigraphic device, providing a dynamic representation of an activity of the PW 4, at a macroscopic level, for example by determining a number of trips, a timeline when the PW 4 is used, distances travelled, speed of travel verses types of environment, community life space, and the like. The processor determines a profile of use of the actigraphic device, and of the PW 4, by using a fusion process to correlate the various acquired parameters, including one or more parameter related to a movement of the actigraphic device and one or more parameter related to the environment of the device. The processor then stores the profile of use in memory and, optionally, some or all of the parameters acquired from the various internal and external sensors. Of course, parameters from a plurality or from all of the sensors, including internal and external sensors, may be correlated for determining the profile of use of the actigraphic device. The profile of use and the parameters may be collected and correlated over time. They may be later post-processed for determining a user profile for a user the PW 4, either within the WIMU-GPS 2 or within an external processing unit (not shown) connected to the WIMU-GPS 2 via the I/O control box 16.

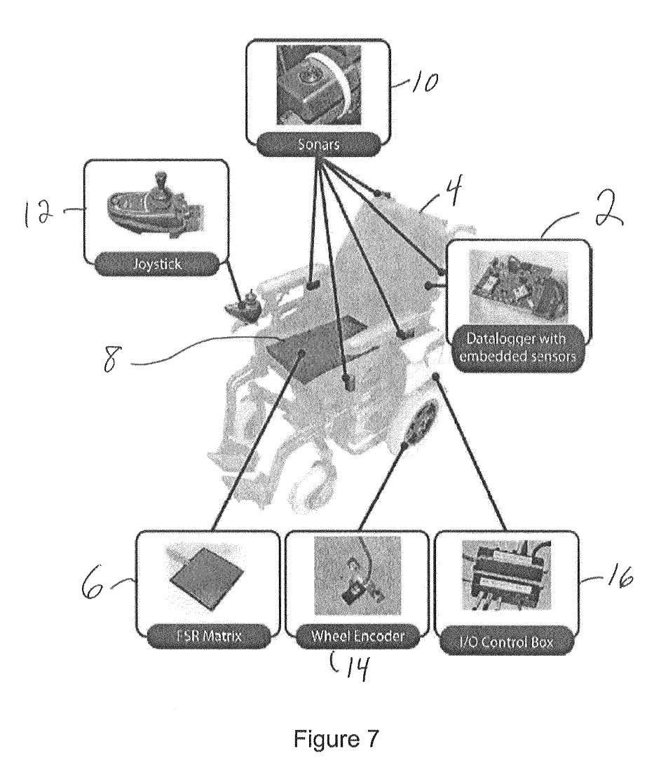

FIG. 8 is a perspective view of the powered wheelchair of FIG. 7, also showing axes for inertial measurements and sample data therefor. The left-hand side of FIG. 8 shows how an IMU may measure angles and spatial orientation of the PW 4 over a 3D space, in terms of yaw, pitch and roll angles. The right-hand side of FIG. 8 shows exemplary sample data, collected over a time axis t, for the yaw angle, the pitch angle, and the roll angle. It may be observed that some sensors may act at once to provide more than one type of parameters. For example, the IMU may act as a motion sensor and detect a forward acceleration and/or a lateral acceleration of the PW 4, providing dynamic mobility parameters of the PW 4, while also acting as an environment sensor by detecting a rapid vertical acceleration of the PW 4, providing a parameter related to the presence of a bump on the ground, this detection reflecting the environment of the PW 4.

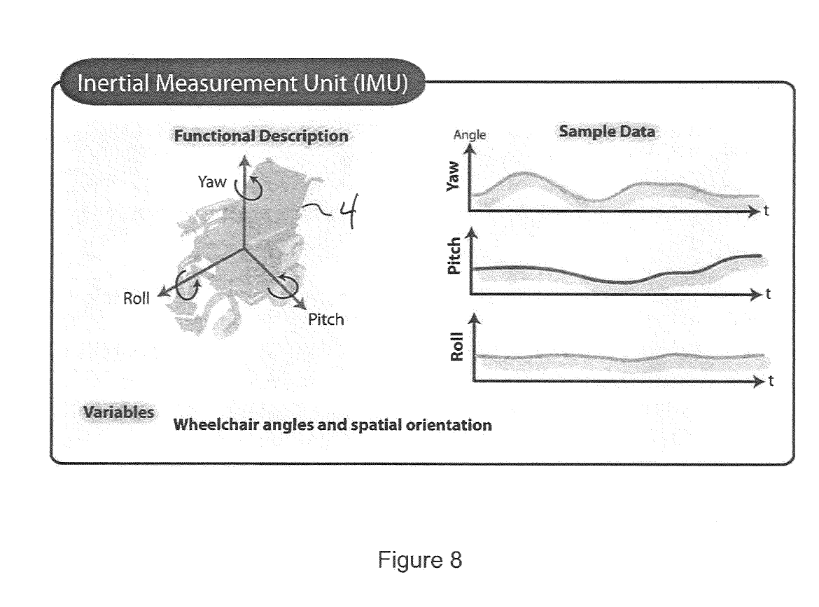

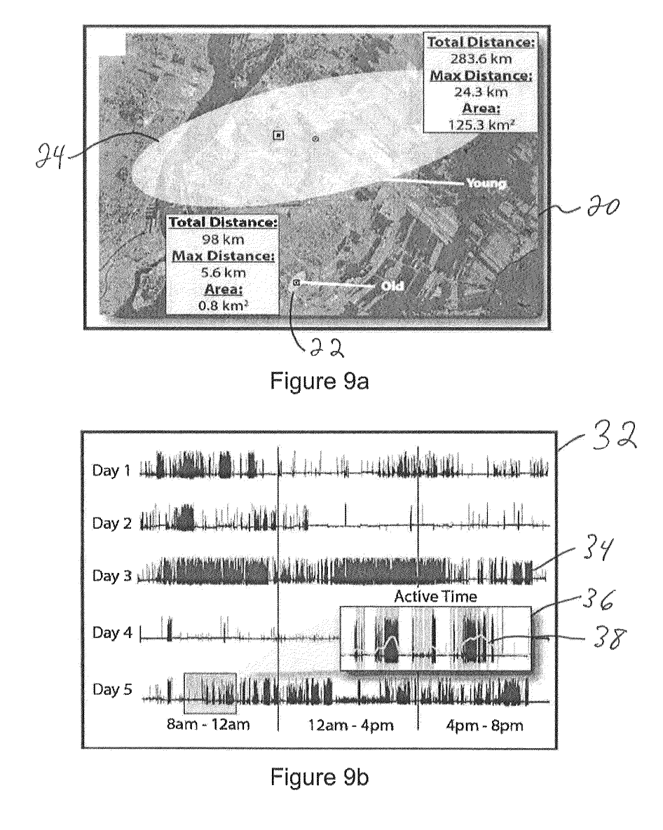

FIG. 9a is a first geographical map showing lifespace data recorded over 5 days. A GPS receiver was used to provide georeference data and lifespace information of the user, allowing to determine hot spots frequently visited by the user. A map 20 is obtained from parameter acquisition and parameter processing, over the 5 day period. The map 20 shows lifespace data from WIMU-GPS data collected over 5 days of recordings for an older adult, 70 years of age, and a younger individual, 30 years of age. A first standard deviational ellipse based on geo-coded data is shown in the form of mobility zones for the older adult, at 22, and for the younger adult, at 24. The total areas in km² of the mobility zones 22 and 24 are based on shown values of total distances travelled over the period and the maximum distance travelled in a single day. Corresponding to the scenario of FIG. 9a, FIG. 9b is graph showing activity data recorded over 5 days. A graph 32 corresponds to the activity of the younger individual as introduced in the description of FIG. 9a. For each Day 1-5, active time and activity counts were computed from integration of 3D accelerometer signals and recorded. A level of activity 34 is shown over periods of time covering a 12-hour overall period. A window 36 provides a zoomed view of a part of the 5^{th} day, highlighting a filtered activity level 38.

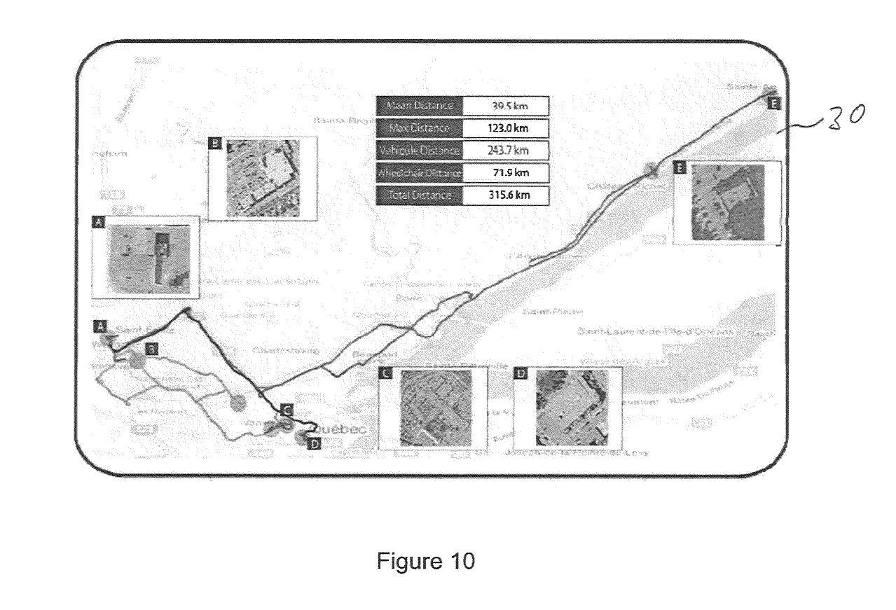

FIG. 10 is a second geographical map showing lifespace data recorded over 7 days. Parameters were recorded and processed with a PW 4 user for 7 days. A map 30 shows trips of the user as derived from the WIMU-GPS data over 7 days, in solid lines. The lifespace corresponds to the area travelled by the user. It is also possible to identify hot spots A-E, corresponding to areas where the user spent a long period. Combining the data obtained by the sonars 10, it is also possible to evaluate the surrounding environment when the PW 4 is in use. Driving behavior should be in tune with the surrounding environments and skill of the users. One may look at exposure to such conditions in the user environment and assess the user skills.

Data from the sensor may also be used to identify specific events of interest, for example battery charging cycles, inclination of the PW 4, unsafe impacts within those recordings. Specific events may be identified using support vector machines (SVMs), neural network, fuzzy logic, or of any similar classification methods for example by combining inputs from numerous sensors. Thereby, user behaviors may be inferred. For example impacts measured with a 3D accelerometer of the WIMU-GPS 2 may be detected and classified using their acceleration magnitude, rated as small, medium, or large. Using the joystick 12 inputs and sonar 10 data before and after the moment of impact allows characterizing the intent of the user and the environment where the PW 4 is in operation. The speed of the PW 4 prior to and after the impact, as measured from the wheel encoder 14, and the displacement of the center of pressure of the user, as measured from the FSR matrix 6, may be used as outcomes of that impact. A large impact recorded a high speed of travel with no changes in direction or speed prior to the impact and a significant acceleration and displacement of the user's center of pressure under the seat may be classified as an unsafe behavior. Repeated small and medium impacts in tight environment may be representative of the skills of the individual in maneuvering the PW. A similar approach may be used to detect other types of events.

Applications

The following section describes a series of applications that are using actigraphic platform and device described hereinabove. This description provides further understanding of the platform, device and method described herein. The following description is exemplary and is not intended to limit the scope of the present disclosure.

The actigraphic device may be expanded with external sensors that may be used to capture information on its surrounding environment. Those sensors may be but are not limited to: ultrasonic range finders (such as sonars), laser range finder, infrared range finder and pyro-electric sensors. By using one or a combination of those sensors, it is possible to capture the presence of objects around the subject or equipment and approximate their distance. For example, by combining ultrasonic range finders such as sonars with an internal GPS, the device may analyze the environment around a user and compute a map of the surrounding at a specific time. That map may be used to correlate to an activity level, to find dangerous behaviors of the user (for example, a user consistently driving a powered wheelchair or a car dangerously close to obstacles) and to characterize its community mobility. A community mobility profile may be cross-correlated with data coming from a geographic map defining location of key areas such as stores, community places and the like, in order to further analyze the behavior and mobility of the user. By combining this community mobility profile with a voice activity detection (VAD) sensor or system, the device may also log the social behavior of the user. Further environmental characterization may be obtained by interfacing pyro-electric sensors on the device, providing information regarding heat around the equipment or the subject wearing the device. That information may be refined with the VAD system and processed to allow for person detection, thus providing another level in social behavior characterization.

The device may also be used as an interface to external devices or equipment useful in many fields. These applications may include, but are not limited to, robotics, automotive driving, powered wheelchair monitoring and assistive devices characterizations. Using the embedded accelerometer, gyroscope and magnetometer, the device may estimate the motion of a mobile robot in order to provide feedback on its position and inclination. That feedback may be used to provide motor commands to control the robot in its environment. When used in automotive driving evaluation, the device may use its internal sensors to compute the lifespace of the user. By externally connecting to a force sensitive sensor installed on the throttle and brake pedals of a car, the device may be used to log and characterize pedals utilization in a real-world application or a simulator. The device, in that context, may also be used as a motion sensor to evaluate the speed and acceleration of the car using the on-board accelerometer and GPS, and thus calculate the active time that may be defined as, for example, the time the car was moving. When combined with a current sensor for example, the device may, be used to characterize the usage of the internal battery pack powering the wheelchair's motors. That information may then be used to optimize the battery charge and discharge cycles. Another application of the device may be in the field of assistive devices such as walking canes on which force sensing resistor or any other pressure sensor may be installed and combined with the internal accelerometer to record the walking pattern of the user.

The WIMU-GPS may serve as a generic data acquisition platform and provide the means to connect various sorts of biometric sensors. The external I/O connectors include analog inputs and digital communication lines (I²C, SPI, UART) and will provide power to almost any external biometric sensors. For example, an oxymeter worn on a finger may be wired to the I/O connector and will send data to a wearable actigraphic device, worn at the forearm, using the UART communication port. The heart rate and oxygen saturation level (SpO₂) will be processed by the onboard microcontroller and will be recorded on the memory card or sent to a remote computer using the onboard Zigbee® radio transmitter. A respiratory belt worn by a patient may also be connected to the analog port of the I/O connector. Respiratory rate and volume will be recorded on the WIMU-GPS as raw data or further processed by the microcontroller to output clinical variables. ECG (electrocardiograph) sensor as well as EMG (electromyograph) may be connected to the I/O port of the WIMU-GPS in order to obtain precise information about the heart rate signals and muscle activation/contraction. The WIMU-GPS may then be worn on the trunk to facilitate wiring. For specific Zigbee® compatible devices, the WIMU-GPS will also accept sensory inputs using the embedded wireless communication module. Finally, all sorts of force sensing devices, such as load cells, strain gauges, and force sensing resistor, may be wired on the analog inputs of the WIMU in order to measure forces, for example plantar pressure, joint forces, and the like.

In the interest of clarity, not all of the routine features of the implementations of the actigraphic platform, device and method are shown and described. It will, of course, be appreciated that in the development of any such actual implementation of the actigraphic device, numerous implementation-specific decisions must be made in order to achieve the developer's specific goals, such as compliance with application-, system-, network- and business-related constraints, and that these specific goals will vary from one implementation to another and from one developer to another. Moreover, it will be appreciated that a development effort might be complex and time-consuming, but would nevertheless be a routine undertaking of engineering for those of ordinary skill in the field of sensor devices, actigraphic devices and, more generally, biomedical devices, having the benefit of this disclosure.

In accordance with this disclosure, the components, process steps, and/or data structures described herein may be implemented using various types of operating systems, computing platforms, network devices, computer programs, and/or general purpose machines. In addition, those of ordinary skill in the art will recognize that devices of a less general purpose nature, such as hardwired devices, field programmable gate arrays (FPGAs), application specific integrated circuits (ASICs), Digital Signal Processors (DSPs) or the like, may also be used. Where a method comprising a series of process steps is implemented by a processor, a computer or a machine and those process steps can be stored as a series of instructions readable by the machine, they may be stored on a tangible medium.

Systems and modules described herein may comprise software, firmware, hardware, or any combination(s) of software, firmware, or hardware suitable for the purposes described herein. Software and other modules may reside on servers, workstations, personal computers, computerized tablets, personal digital assistants (PDAs), and other devices suitable for the purposes described herein. Software and other modules may be accessible via local memory, via a network, via a browser or other application in an application service provider (ASP) context, or via other means suitable for the purposes described herein. Data structures described herein may comprise computer files, variables, programming arrays, programming structures, databases or any electronic information storage schemes or methods, or any combinations thereof, suitable for the purposes described herein.

Although the present disclosure has been described hereinabove by way of non-restrictive illustrative embodiments thereof, these embodiments can be modified at will within the scope of the appended claims without departing from the spirit and nature of the present disclosure.

What is claimed is:

1. An actigraphic device, comprising:
a motion sensor for detecting a parameter related to a movement of the device;
an environment sensor for detecting a parameter related to an environment of device;
a processor for determining a profile of use of the device by correlating the parameter related to the movement of the device and the parameter related to the environment of the device; and
a memory for recording the profile of use.

2. The device of claim 1, wherein the device is mounted on a vehicle.

3. The device of claim 2, wherein the vehicle is selected from the group consisting of a wheelchair, a mobile robot and an automobile.

4. The device of claim 2, comprising:
a user behavior sensor for detecting a behavioral parameter of a user of the device;
wherein the processor is further for correlating the behavioral parameter with the parameter related to the movement of the device and the parameter related to the environment of the device.

5. The device of claim 4, wherein:
the user behavior sensor is selected from the group consisting of a joystick, a pedal, an actuator, a steering wheel, a force sensing resistor and any combination thereof.

6. The device of claim 1, wherein:
the environment sensor is selected from the group consisting of a sonar, a camera, an accelerometer, a GPS receiver, a gyroscope, a magnetometer, a voice activity detector, and any combination thereof.

7. The device of claim 1, wherein:
the motion sensor is selected from the group consisting of a GPS receiver, an accelerometer, a gyroscope, a magnetometer, and any combination thereof.

8. The device of claim 1, comprising:
a post-processor for determining a user profile for a user of the device based on instances of the profile of use collected over time.

9. The device of claim 1, wherein the parameter related to an environment of the device is for indicating a location of the device.

10. The device of claim 1, comprising:
a fusion process for mitigating random variations in the detected parameters.

11. The device of claim 1, comprising:
a communication port for outputting the profile of use, the parameter related to the movement of the device and the parameter related to the environment of the device.

12. The device of claim 11, wherein:
the communication port is adapted for wireless communication use.

13. The device of claim 1, comprising:
a communication port for inputting an external sensor parameter;
wherein the processor is further for correlating the external sensor parameter with the parameter related to the movement of the device and the parameter related to the environment of the device.

14. The device of claim 1, wherein:
the parameter related to the movement of the device is determined in a three-dimensional space.

15. The device of claim 1, further comprising:
a biometric sensor for detecting a physical parameter of a user;
wherein the processor is further for correlating the physical parameter with the parameter related, to the movement of the device and the parameter related to the environment of the device.

16. The device of claim 1, wherein the device is wearable by a user.

17. The device of claim 16, wherein the device is adapted for forming a network with at least another actigraphic device.

18. The device of claim 17, wherein the network comprises:
a first actigraphic device comprising an output port for outputting a first profile of use, and
a second actigraphic device comprising an input port for receiving the first profile of use,
wherein the second actigraphic device is further for producing a second profile of use, and
wherein a processor of the second actigraphic device is for determining a combined profile of use by correlating the first profile of use and the second profile of use.

19. The device of claim 18, wherein:
the first and second actigraphic devices are for communicating via a wired link.

20. The device of claim 18, wherein:
the first and second actigraphic devices are for communicating via a wireless link.

21. The device of claim 18, wherein:
the first and second actigraphic devices are for positioning on two distinct body segments of the user.

22. A method of acquiring a profile of use of an actigraphic device, comprising:
detecting a parameter related to a movement of the device;
detecting a parameter related to an environment of the device;
determining the profile of use of the device by correlating the parameter related to the movement of the device and the parameter related to the environment of the device; and
storing the profile of use in a memory.

23. The method of claim 22, further comprising:
receiving data from an external sensor at the device;
correlating the received data with the parameter related to the movement of the device and the parameter related to the environment of the device.

24. The method of claim 22, wherein:
at least one of the parameters is detected by an element selected from the group consisting of a GPS receiver, an accelerometer, a gyroscope, a magnetometer, and any combination thereof.

25. The method of claim 22, further comprising:
outputting the profile of use towards a post-processing device.

26. The method of claim 22, comprising:
determining a user profile of a user of the device based on instances of the profile of use collected over time.
